(19) United States
(12) Patent Application Publication
(10) Pub. No.: US 2008/0281235 A1
(43) Pub. Date: Nov. 13, 2008

(54) Angular Displacement Sensor for Joints And Associated System and Methods

(76) Inventor: David J. Cowin, Windermere, FL (US)

Correspondence Address: JACQUELINE E. HARTT, PH.D;ALLEN, DYER, DOPPELT, MILBRATH & GILCHRIST, P.A., P.O. BOX 3791, ORLANDO, FL, 32802-3791, US

(21) Appl. No.: 12/121,959

(22) Filed: May 16, 2008

Related U.S. Application Data
(63) Continuation-in-part of application No. 11/833,296, filed on Aug. 3, 2007.
(60) Provisional application No. 60/821,463, filed on Aug. 4, 2006.

(51) Int. Cl. A61B 5/117

(52) U.S. Cl. 600/595

(57) ABSTRACT

A system for monitoring joint position following introduction of a joint prosthesis in a patient includes a first angular movement sensor positioned adjacent a first side of a bodily joint of a patient and a second angular movement sensor positioned adjacent a second, opposite side of the bodily joint. A receiver can receive data from the angular movement sensors. A processor in signal communication with the receiver has a comparator for determining whether the received data from the sensors indicate that movement of the bodily joint is less than a predetermined limit. An indicator in signal communication with the comparator is provided for warning the patient if the joint movement is determined by the comparator to be above the predetermined limit. The received data can be tracked over time.

### CROSS-REFERENCE TO RELATED APPLICATIONS

This application is a continuation-in-part of application Ser. No. 11/833,296, filed Aug. 3, 2007, which itself claims priority to provisional application Ser. No. 60/821,463, filed Aug. 4, 2006.

### BACKGROUND OF THE INVENTION

1. Field of the Invention

The present invention relates to devices and methods for protecting against joint displacement, and, more particularly, to such devices and methods for protecting against hip displacement in patients who have undergone a hip or other joint replacement procedure.

2. Description of Related Art

When a patient has undergone a total hip arthroplasty, the risk of hip dislocation is in the range of 1-5%, with a higher risk present with revisions to the procedure. A reason for this risk is that the patient's proprioception of the joint is lost with the hip replacement, and thus the patient cannot sense when the joint is approaching a dangerous position. Therefore, the patient may not realize that the hip is about to be dislocated until it is too late. Some methods currently being used include braces to prevent excessive movement, but these are bulky and uncomfortable and do not train the patient not to exceed movement limits once the brace is removed.

Therefore, it would be desirable to provide a device and method for indicating to a patient when a safe range of hip movement is being exceeded, and also to provide biofeedback so that the patient ultimately learns the limits of the safe range without such an indication.

Another need exists in the art for monitoring a patient's movement during a healing period. Such a monitoring could assist the physician in tracking progress with actual displacement readings instead of relying on the patient to self-report. Such data could also be useful in cases wherein the patient's progress needs to be reported to an outside agency, such as for worker's compensation claims or other legal reasons.

Such a device and method would also be applicable to other joint displacement sensing, prevention, and monitoring.

### SUMMARY OF THE INVENTION

A system is provided for preventing joint displacement in a patient. The system can comprise a first angular movement sensor that is affixed on a first side of a bodily joint of a patient and a second angular movement sensor that is affixed on a second side of the bodily joint of the patient. The second side is on an opposite side of the joint from the first side.

A receiver is provided that is adapted to receive data from the first and the second angular movement sensors. A comparator that is in signal communication with the receiver can determine whether the received data from the first and the second angular movement sensors indicate that movement of the bodily joint is within predetermined limit. An indicator in signal communication with the comparator is adapted to warn the patient if the joint movement is determined by the comparator to be outside the predetermined limits.

In another embodiment, a system and method are provided for monitoring joint position following introduction of a joint prosthesis in a patient. The system comprises a first angular movement sensor implanted adjacent a first side of a bodily joint of a patient and a second angular movement sensor implanted adjacent a second side of the bodily joint of the patient, the second side on an opposite side of the bodily joint from the first side. The first and the second angular movement sensors each have a power source that is rechargeable from exterior the bodily joint.

A receiver is adapted to receive data from the first and the second angular movement sensors. A processor in signal communication with the receiver has comparison means resident thereon for determining whether the received data from the first and the second angular movement sensors indicate that movement of the bodily joint is within predetermined limits. An indicator in signal communication with the comparison means is provided for warning the patient if the joint movement is determined by the comparison means to be outside the predetermined limits.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWINGS

FIG. 1 is a schematic diagram of sensor placement on a patient's body.

FIG. 2 is an exemplary control device for setting the range of the sensors.

FIG. 3 is a schematic diagram of an embodiment wherein the sensors are implanted.

FIG. 4 is a system schematic for recording sensor output over time.

FIG. 5 is a schematic diagram of an embodiment wherein the sensors are insertable into a garment.

### DETAILED DESCRIPTION OF THE PREFERRED EMBODIMENTS

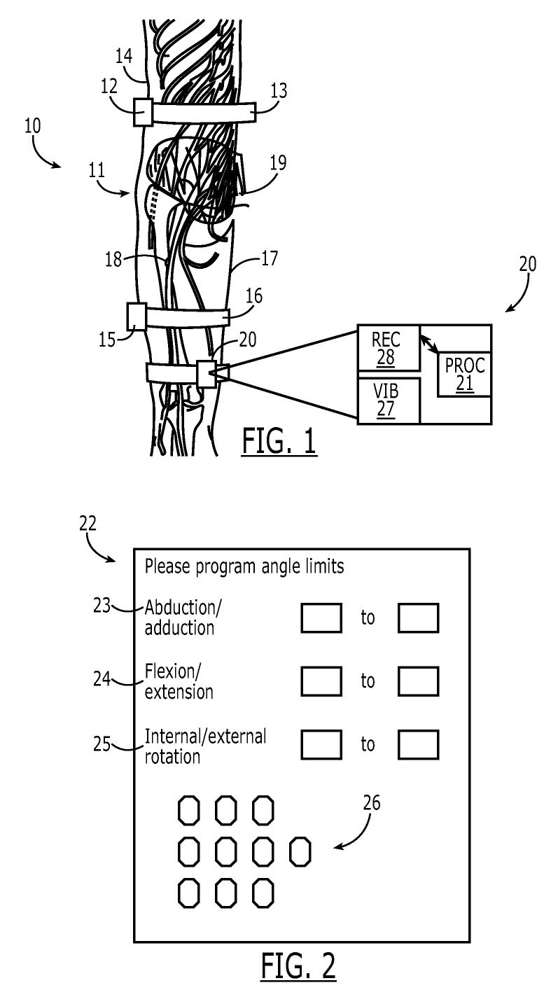

A description of the preferred embodiments of the present invention will now be presented with reference to FIGS. 1-5. Although the following is presented in terms of an embodiment for use with the hip, it will be understood by one of skill in the art that the system could be used with any bodily joint on any creature with a skeletal system.

In an embodiment, a system 10 (FIG. 1) is provided for preventing joint displacement in a patient, here, for use with the hip 11 of a patient who has, for example, undergone a total hip replacement. The system 10 can comprise a first angular movement sensor 12 that is affixed above the hip 11, such as affixed to a band 13 or girdle worn about the patient's waist 14. A second angular movement sensor 15 is affixed below the hip 11, for example, affixed to a band 16 wrapped about the patient's thigh 17. The sensors 12,15 can each comprise, for example, a battery-powered gyroscope capable of wireless communication, although this is not intended as a limitation. The sensors 12,15 can communicate with each other so that the three-dimensional angular position of one device relative to the other can be determined. This angular position corresponds to the position of the femur 18 relative to the pelvis 19 at the hip joint 11.

In a particular embodiment, a device 20 containing a processor 21 and a receiver 22 is provided that is adapted to receive data from the angular movement sensors 12,15. The processor 21 is programmable, for example, via a signal generated by a handheld input device 22 such as that illustrated in FIG. 2, to contain a set of angular limits to which the joint should be subjected. In the unit 22 shown, for example, the angle limits of abduction/adduction 23, flexion/extension 24, and internal/external rotation 25 can be set, for example, by a physician, via a numerical keypad 26 or other type of input method, such as touch screen and stylus, up/down keys, etc.

The processor 21 can then determine from the received data from the angular movement sensors 12,15 whether movement of the hip 11 is within the predetermined limits.

An indicator in signal communication with the device 20 is in signal communication is adapted to warn the patient if the hip movement is approaching or outside the predetermined limits. Such an indicator can comprise, for example, a vibrator 27 that is affixable to the patient's body to provide biofeedback limiting hip movement. In a particular embodiment, the intensity of the vibration can be indicative of the closeness to the predetermined limits. One of skill in the art will recognize that other types of indicators can be used instead of or in concert with a vibrator, for example, an audio signal. Such a biofeedback system will teach the patient the acceptable ranges of movement and ultimately will be unnecessary.

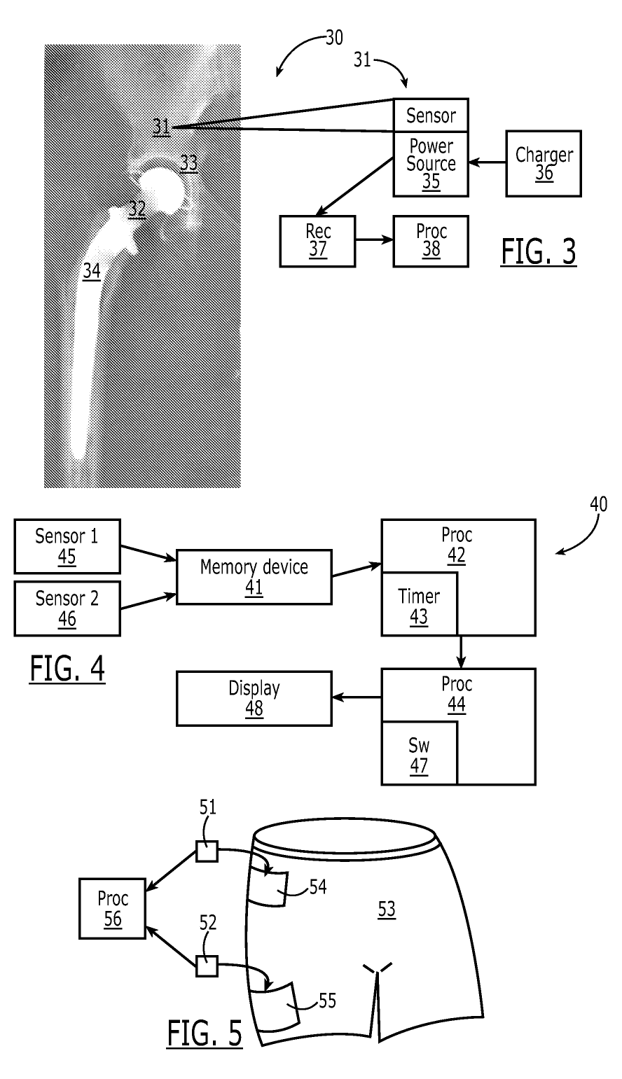

In another embodiment of the system 30 (FIG. 3), the angular movement sensors 31,32 are implantable in the patient's body, for example, on the hip 33 and in the prosthesis 34 itself. Each of the sensors 31,32 includes a power source 35 that is rechargeable with an induction charger 36 from exterior the patient. Such sensors 31,32 are preferably very small, for example, 3 mm in their largest dimension, and can transmit wirelessly to a receiver 37, which can transmit sensor data to a processor 38 as discussed above with reference to the first embodiment 10.

In a further embodiment 50 (FIG. 5) the sensors 51,52 can be inserted into a wearable garment 53. In the particular embodiment shown, the garment 53 comprises a pair of stretchable shorts, similar to bicycle shorts, for use in monitoring hip movement. This embodiment 50 is not intended to be limiting, and one of skill in the art will appreciate that other types of garments for use in monitoring other joints are also contemplated in this invention. For example, a pair of long pants could be used to monitor knee or ankle movement; a shirt could be used to monitor elbow, neck, or shoulder movement.

The garment 53 has at least two pockets 54,55 dimensioned and positioned for holding the sensors 51,52 therein, the sensors 51,52 capable of communication with a processor 56 for joint angle monitoring in similar fashion to the embodiments 10,30 discussed above.

Yet another aspect of the invention is directed to a system 40 (FIG. 4) for monitoring the patient's joint position over time. In this embodiment 40 is included a memory device 41 in signal communication with the processor 42, which further has a timing element 43 associated therewith. Another processor 44 can read sensor 45,46 data from the memory device 41, which can be processed using software 47 for displaying 48 a time history of the joint movement. The display 48 can comprise, for example, a monitor or a printer, for displaying a chart, graph, or spreadsheet, or other data display output such as known in the art.

Such a display can be used to monitor the patient's joint movement, thereby avoiding reliance on self-reporting, to provide an accurate indicator of progress. Such data can be useful to the physician, and also to agencies charged with monitoring patient progress for such reasons as worker's compensation claims or liability claims.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the apparatus illustrated and described herein are by way of example, and the scope of the invention is not limited to the exact details of construction.

What is claimed is:

1. A system for monitoring joint position following introduction of a joint prosthesis in a patient, the system comprising:
a first angular movement sensor positionable adjacent a first side of a bodily joint of a patient;
a second angular movement sensor positionable adjacent a second side of the bodily joint of the patient, the second side on an opposite side of the bodily joint from the first side;
a receiver adapted to receive data from the first and the second angular movement sensors;
a processor in signal communication with the receiver having comparison means resident thereon for determining whether the received data from the first and the second angular movement sensors indicate that movement of the bodily joint is less than a predetermined limit; and
an indicator in signal communication with the comparison means forwarning the patient if the joint movement is determined by the comparison means to be above the predetermined limit.

2. The system recited in claim 1, wherein the prosthesis introduction comprises a hip replacement, the hip first side comprises a location on a pelvis of the patient, and the hip second side comprises a location on the hip prosthesis.

3. The system recited in claim 1, wherein the first and the second angular movement sensor each comprise a battery-powered gyroscope adapted for wireless communication with the receiver.

4. The system recited in claim 1, further comprising an input device adapted for signal communication with the processor, the input device comprising means for receiving the predetermined angular movement limit from a user and for transmitting the received predetermined angular movement limit to the processor.

5. The system recited in claim 4, wherein the input device comprises a handheld input device comprising means for entering angular limits of abduction/adduction, flexion/extension, and internal/external rotation.

6. The system recited in claim 5, wherein the entering means comprises a numerical keypad.

7. The system recited in claim 1, wherein the indicator comprises one of a vibrator affixable to a body of the patient and a means for issuing an audio signal.

8. The system recited in claim 1, wherein the indicator comprises means for issuing a warning signal to the patient, and wherein an intensity of an issued warning signal is related to a difference between the predetermined limit and the received data from the first and the second angular movement sensors.

9. The system recited in claim 1, wherein the processor further has a timing element, and further comprising a memory device in signal communication with the processor, for storing a time history of the received data from the first and the second angular movement sensors.

10. The system recited in claim 9, further comprising a reading device for downloading the stored time history from the memory device thereinto, for use in tracking the movement of the bodily joint over time.

11. The system recited in claim 1, wherein the first and the second angular movement sensors are implantable within the patient.

12. The system recited in claim 11, wherein the second angular movement sensor is implantable within the prosthesis.

13. The system recited in claim 11, wherein the first and the second angular movement sensors each have a power source that is rechargeable from exterior the bodily joint, and further comprising an induction charger for recharging the power sources of the first and the second angular movement sensors.

14. The system recited in claim 1, further comprising a garment wearable by the patient, the garment having a pocket dimensioned and positioned for retaining the first and the second angular movement sensor therein.

15. The system recited in claim 14, wherein the joint prosthesis comprises a hip replacement prosthesis, and the garment comprises a pair of shorts.

16. The system recited in claim 14, wherein the joint prosthesis comprises at least one of a shoulder replacement and an elbow replacement, and the garment comprises a shirt.

17. The system recited in claim 14, wherein the joint prosthesis comprises at least one of a knee replacement and an ankle replacement, and the garment comprises a pair of long pants.

18. A method for monitoring joint position following introduction of a joint prosthesis in a patient, the method comprising:
sensing a first angular movement using a first sensor positioned adjacent a first side of a bodily joint of a patient;
sensing a second angular movement using a second sensor positioned adjacent a second side of the bodily joint of the patient, the second side on an opposite side of the bodily joint from the first side;
receiving data from the first and the second sensors;
determining whether the received data indicate that movement of the bodily joint is less than a predetermined limit; and
warning the patient if the joint movement is determined to be above the predetermined limit.

19. The method recited in claim 18, wherein the prosthesis introduction comprises a hip replacement, the hip first side comprises a location on a pelvis of the patient, and the hip second side comprises a location on the hip prosthesis.

20. The method recited in claim 18, wherein the first and the second angular movement sensing is performed with the use of a battery-powered gyroscope adapted for wireless communication.

21. The method recited in claim 18, further comprising, prior to the determining step, entering the predetermined angular movement limit into an input device.

22. The method recited in claim 21, wherein the entering step comprises entering angular limits of abduction/adduction, flexion/extension, and internal/external rotation.

23. The method recited in claim 18, wherein the warning step comprises issuing a signal, an intensity of the issued warning signal related to a difference between the predetermined limit and the received data from the first and the second angular movement sensing steps.

24. The method recited in claim 18, further comprising storing a time history of the received data from the first and the second sensors.

25. The method recited in claim 24, further comprising downloading the stored time history to a processor and using the downloaded time history to track the movement of the bodily joint over time.

26. The method recited in claim 18, wherein the first and the second angular movement sensors are implanted.

27. The method recited in claim 26, wherein the second angular movement sensor is implanted within the prosthesis.

28. The method recited in claim 18, wherein the first and the second sensors each have a power source that is rechargeable from exterior the bodily joint, and further comprising recharging the power sources of the first and the second sensors using induction charging.

29. The method recited in claim 18, further comprising, prior to the sensing steps, placing a garment on the patient, the garment having a pocket dimensioned and positioned for retaining the first and the second angular movement sensor therein, and placing the first and the second angular movement sensors in the pocket.
